(19) United States
(12) Patent Application Publication
(10) Pub. No.: US 2024/0335497 A1
(43) Pub. Date: Oct. 10, 2024

(54) PULSE PROTEIN-BASED COMPOSITION FOR ACTIVATING THE SYNTHESIS OF FGF19

(71) Applicant: ROQUETTE FRERES, Lestrem (FR)

(72) Inventors: Catherine LEFRANC-MILLOT, Lestrem (FR); Hubert VIDAL, Pierre Bénite (FR); Clémence DEFOIS, Pierre Bénite (FR)

(21) Appl. No.: 18/291,488

(22) PCT Filed: Jul. 26, 2022
(86) PCT No.: PCT/EP2022/025353

§ 371 Date: Jan. 23, 2024

(30) Foreign Application Priority Data
Jul. 26, 2021 (FR) 2108059

(51) Int. Cl. A61K 38/01; A23J 3/14; A61K 31/42; A61K 31/575; A61K 36/48

(52) U.S. Cl. CPC A61K 38/011; A23J 3/14; A61K 31/42; A61K 31/575; A61K 36/48

(57) ABSTRACT

A pulse protein composition, preferably of pea or faba bean, the degree of hydrolysis of which is less than 10%, in particular for therapeutic use, preferably in the prevention and/or treatment of a disease susceptible to treatment by activation of the synthesis of FGF19.

### TECHNICAL FIELD

The present invention is a legume protein composition, preferentially of pea or faba bean, the degree of hydrolysis of which is less than 10%, in particular for therapeutic use, preferably in the prevention and/or treatment of a disease susceptible to treatment by activation of the synthesis of FGF19.

### PRIOR ART

A fibroblast is a human cell that is present in connective tissue. The most important role of fibroblasts is to maintain the extracellular matrix of the connective tissues, and to repair lesions due to trauma. They also serve to regulate the organization and differentiation of the cells of the surrounding tissues. These fibroblasts, inter alia, secrete the extracellular matrix, that is to say the proteins which form the fibers of the connective tissue and the glycoproteins of the ground substance. They also participate in the metabolism of lipoproteins (LDL) and cholesterol.

Fibroblast Growth Factors (FGF) form a family comprising 23 proteins that have been identified to date in humans (FGF1, FGF2, . . . FGF23), which activate the migration and multiplication of fibroblasts. These factors are generally secreted by fibroblasts.

FGF19 (Fibroblast Growth Factor 19) is a protein belonging to this family of fibroblast growth factors. Its synthesis is activated by the FXR (Farnesoid X Receptor), which itself is stimulated by bile salts, which has the consequence of inhibiting gluconeogenesis and the synthesis of bile salts and of activating the synthesis of proteins and glycogen. It also acts on the metabolism of lipids and calcium.

In humans, a low serum content of FGF19 is associated with more serious forms of non-alcoholic steatohepatitis or NASH (Alisi A, Ceccarelli S, Panera N et al. Association between serum atypical fibroblast growth factors 21 and 19 and pediatric nonalcoholic fatty liver disease, PLOS One, 2013;8:e6716). Its use to combat NASH was therefore studied. On an animal model, it unfortunately appears to promote the formation of hepatocellular carcinoma. An analog of the molecule was developed, without oncogenesis activity. It appears to be promising in the treatment of non-alcoholic steatohepatitis, reducing the lipid content of hepatic cells. However, the use of an analog is constraining due to the need for exogenous synthesis of said analog and its consumption as a drug. There is therefore an unmet need to naturally activate the in vivo synthesis of FGF19 to prevent or treat NASH.

Sarcopenia is a geriatric syndrome, which is initially characterized by a reduction in muscle capacities due to age and which, as it worsens, causes a deterioration of muscle strength and physical performance. Sarcopenia observed in elderly persons can be attributed to the aging process but can be accelerated by pathological and behavioral factors such as poor nutrition and a sedentary lifestyle. FGF19 has recently appeared as an opportunity for combatting the prevalence of sarcopenia (“Fibroblast growth factor 19 regulates skeletal muscle mass and ameliorates muscle wasting in mice.” Benoit & al., Nat Med. 2017 Aug;23(8):990-996). As in the treatment of NASH, there is therefore an unmet need to naturally activate the in vivo synthesis of FGF19 in order to combat the prevalence of this syndrome.

### SUMMARY

It is to the applicant's credit to have demonstrated that a composition of legume proteins, particularly of slightly hydrolyzed pea proteins or non-hydrolyzed faba bean proteins, made it possible to activate the in vitro synthesis of FGF19 in human cell cultures, in particular when these proteins are the only protein source. Ingesting them daily in the daily food intake therefore makes it possible to envisage naturally overexpressing the synthesis of FGF19 and therefore to combat the prevalence of or treat syndromes such as NASH or sarcopenia, cited herein in a preferential and non-exhaustive manner.

The present invention is therefore firstly a composition of legume proteins, preferentially of pea or faba bean, the degree of hydrolysis of which is less than 10%, for therapeutic use in a subject in need thereof, preferably for use in the prevention and/or treatment of a disease susceptible to treatment by activation of the synthesis of FGF19, especially a disease associated with dysregulation of the FGF19 signaling pathway.

The present invention is also a pharmaceutical composition intended to overexpress FGF19 comprising legume proteins, preferentially of pea or faba bean, the degree of hydrolysis of which is less than 10%, and optionally a pharmaceutically acceptable excipient.

The invention will be better understood upon reading the following detailed description

### BRIEF DESCRIPTION OF THE DRAWINGS

FIG. 1 shows the effects of the various digestates on the expression of FGF19. The HT29 cells were exposed to the various protein digestates (PD) alone or in combination with an FXR agonist.

FIG. 2 shows the increase in the expression of FGF19 mRNA following the treatment of HT29 cells with the RQ3 and RQ7 digestates. The HT29 cells were exposed to 0.1, 0.5 and 1 μM GW4064 and to a natural FXR agonist, 50 μM chenodeoxycholic acid (CDCA) alone or in combination for 6 h with the RQ3 or RQ7 extracts.

FIG. 2b shows the increase in the production and the secretion of the FGF19 protein following the treatment of HT29 cells with the RQ3 and RQ7 digestates. The HT29 cells were exposed to 0.1, 0.5 and 1 μM GW4064 and to a natural FXR agonist, 50 μM chenodeoxycholic acid (CDCA) alone or in combination for 6 h (left) and 24 h (right) with the RQ3 or RQ7 extracts.

FIG. 3 shows the increase in the level of expression of FGF19 (mRNA) following the co-culture treatment of Caco-2 and HT29-MTX cells with RQ3 and RQ7 digestates.

### DETAILED DESCRIPTION

### Leguminous Protein Composition According to the Invention

The inventors have shown that a composition of legume proteins, preferably of pea or faba bean, the degree of hydrolysis of which is less than 10%, made it possible to activate the expression of FGF19 in human cells.

The present invention is therefore firstly a composition of legume proteins, preferably of pea or faba bean, the degree of hydrolysis of which is less than 10%, for therapeutic use in a subject in need thereof, preferably for use in the prevention and/or treatment of a disease susceptible to treatment by activation of the synthesis of FGF19, especially a disease associated with dysregulation of the FGF19 signaling pathway.

The term “protein” or “polypeptide” should be understood in the present application to mean the macromolecules formed from one or more polypeptide chains consisting of a sequence of amino acid residues bonded to one another by peptide bonds.

“Leguminous plant” or “legume” will be understood in the present application to mean the family of dicotyledonous plants of the Fabales order. This is one of the largest flowering plant families, third after Orchidaceae and Asteraceae in terms of number of species. It contains approximately 765 genera, bringing together more than 19,500 species. Several leguminous plants are important crop plants, including soy, beans, peas, chickpeas, faba beans, peanuts, cultivated lentils, cultivated alfalfa, various clovers, broad beans, carob and licorice.

In the particular context of pea proteins, the present invention relates more particularly to globulins (approximately 50-60% of the proteins of the pea) and albumins (20-25%). Pea globulins are mainly subdivided into three sub-families: legumins, vicilins and convicilins.

The proteins extracted from these leguminous plants belong predominantly to the sub-groups of the globulins and albumins. In the present invention, the legume protein consists predominantly of globulins; in particular, it contains more than 90% by weight of globulins relative to the total weight of the proteins. Globulins can be distinguished from albumins by various methods well known to those skilled in the art, in particular by their solubility in water, with albumins being soluble in pure water, whereas globulins are only soluble in salt water. It is also possible to identify the albumins and globulins present in a mixture by electrophoresis or chromatography. A preferred method is described in the article “Peptide and protein molecular weight determination by electrophoresis using a high-molarity tris buffer system without urea.” Fling SP, Gregerson DS, Anal. Biochem. 1986;155:83-88.

The legume protein according to the invention contains more than 90% by weight of globulins relative to the total weight of the proteins.

The term “pea” is considered here in its broadest accepted use and includes in particular all the varieties of “smooth pea” and “wrinkled pea” and all the mutant varieties of “smooth pea” and “wrinkled pea”, regardless of the uses for which said varieties are usually intended (human food, animal feed and/or other uses).

The term “peas” in the present application includes pea varieties belonging to the Pisum genus and more particularly to the species sativum and aestivum. Said mutant varieties are especially those called “r mutants”, “rb mutants”, “rug 3 mutants”, “rug 4 mutants”, “rug 5 mutants” and “lam mutants” as described in the article by C-L Heydley et al., entitled “Developing novel pea starches”, Proceedings of the Symposium of the Industrial Biochemistry and Biotechnology Group of the Biochemical Society, 1996, pp. 77-87.

“Faba bean” is understood as the group of annual plants of the species Vicia faba, belonging to the group of leguminous plants of the family Fabaceae, subfamily Faboideae, and tribe Fabeae. A distinction is made between Minor and Major varieties. In the present invention, wild-type varieties and those obtained by genetic engineering or varietal selection are all excellent sources.

Preferably, the legume proteins, preferentially of pea or faba bean, having a degree of hydrolysis of less than 10%, are the only protein source within the composition. Indeed, it is well known in the field of the invention that legume proteins are characterized by an amino acid profile that is deficient in certain amino acids (methionine and cysteine) when compared with the profiles required in human nutrition. This deficiency is often overcome by mixing them with other protein sources such as animal proteins or proteins from grains such as rice or wheat. Mention may be made, for example, of “Protein content and amino acid composition of commercially available plant-based protein isolates” (Gorissen & al., Amino Acids 50, 1685-1695, 2018).

In this preferred embodiment, however, the Applicant aims to maximize the effect of the legume proteins, preferentially of pea or faba bean, having a degree of hydrolysis of less than 10%, present in the composition in order to overexpress FGF19 as much as possible. Any attempt to mix with other protein sources in order to restore the amino acid profile will result in lowering the effect on the overexpression of FGF19.

The degree of hydrolysis may be determined by measuring the content of free amino nitrogen with respect to the total nitrogen, according to the test of the degree of hydrolysis described hereinafter. Its principle consists firstly in determining the content of amino nitrogen (free NH₂) on the sample of proteins using the MEGAZYME kit (reference K-PANOPA), followed by determining the content of protein nitrogen (total nitrogen) of the sample, and finally calculating the degree of hydrolysis using these two contents.
Determining the content of amino nitrogen:

The “amino nitrogen” groups of the free amino acids in the sample react with the N-acetyl-L-cysteine and ophthaldialdehyde (OPA) to form isoindole derivatives.

The amount of isoindole formed during this reaction is stoichiometric with the amount of free amino nitrogen. It is the isoindole derivative which is measured by the increase in absorbance at 340 nm.

A test specimen P*, exactly weighed, of the sample to be analyzed is introduced into a 100 ml beaker. This test specimen will be from 0.5 to 5.0 g based on the amino nitrogen content of the sample. Approximately 50 ml of distilled water is added, homogenization is carried out and the mixture is decanted into a 100-ml graduated flask. 5 ml of 20% sodium dodecyl sulfate (SDS) are added, and the mixture is supplemented with distilled water to reach a volume of 100 ml. Stirring is carried out for 15 minutes with a magnetic stirrer at 1000 rpm. A solution no. 1 is prepared by dissolving a tablet from bottle 1 of the Megazyme kit in 3 ml of distilled water and stirring is carried out until it is completely dissolved. It is necessary to provide one tablet per test. Solution no. 1 is prepared immediately before use.

A blank, a standard and a sample are prepared directly in the cuvettes of the spectrophotometer under the following conditions:
blank: introduce 3.00 ml of solution no. 1 and 50 μl of distilled water
standard: introduce 3.00 ml of solution no. 1 and 50 μl of bottle 3 of the Megazyme kit
sample: introduce 3.00 ml of solution no. 1 and 50 μl of the sample preparation.

The content of each cuvette is mixed and the measure of absorbance (A1) of the solutions is taken after approximately 2 mn in the spectrophotometer at 340 nm (spectrophotometer equipped with cuvettes with 1.0 cm of optical path, able to measure at a wavelength of 340 nm, and verified according to the procedure described in the related manufacturer's technical manual).

The reactions are then immediately initiated by adding 100 μl of solution no. 2, which corresponds to the OPA solution of bottle 2 of the Megazyme kit, to each spectrophotometer cuvette.

The content of each cuvette is mixed and they are then placed in darkness for approximately 20 minutes.

The measure of absorbance A2 of the blank, the standard and the sample are then taken from the spectrophotometer at 340 nm.

The free amino nitrogen content, expressed as a percentage by weight with respect to the weight of the product, is given by the following formula:

$$\% amino nitrogen = \frac{( Δ Aech - Δ Ablc ) \times 3.15 \times 14.01 \times V \times 100}{6803 \times 0.05 \times m \times 1000} \\ \% amino nitrogen = \frac{( Δ Aech - Δ Ablc ) \times 12.974 \times V}{m \times 1000} \qquad [ Math 1 ]$$

wherein:
wherein:
ΔAech=Aech2−Aech1
ΔAblc=Ablc2−Ablc1
Aech2=absorbance of the sample after adding solution no. 2
Aech1=absorbance of the sample after adding solution no. 1
Ablc2=absorbance of the blank after adding solution no. 2
Ablc1=absorbance of the blank after adding solution no. 1
V=volume of the flask
m=weight of the test specimen in g
6803=extinction coefficient of the isoindole derivative at 340 nm (in I.mol⁻¹·cm⁻¹). 14.01=molar mass of the nitrogen (in g·mol⁻¹)
3.15=final volume in the cuvette (in ml)
0.05=test specimen in the cuvette (in ml)
Determining the content of protein nitrogen:

The content of protein nitrogen is determined according to the DUMAS method according to standard ISO 16634-2016. It is expressed as percentage by weight relative to the weight of the product.
Calculating the degree of hydrolysis

The degree of hydrolysis (DH) is calculated with the following formula:

$$DH = \frac{\% amino nitrogen}{\% protein nitrogen} \times 100 \qquad [ Math 2 ]$$

As will be demonstrated hereinafter in the section relating to the examples of this patent application, it is important within the meaning of the invention to use pea proteins that have a degree of hydrolysis strictly between 5% and 10%. Indeed, if the degree of hydrolysis is higher or lower, it is observed that the effect of activating the synthesis of FGF19 is less effective.

### Method for Preparing Legume Proteins According to the Invention

In a first preferential embodiment of the invention, the legume proteins are pea proteins, the degree of hydrolysis of which is preferentially between 5% and 10%.

Pea proteins, having a degree of hydrolysis strictly between 5% and 10%, are obtained by any means well known to a skilled person.

The starting base is a flour, preferentially a concentrate (solids more than 50% protein), even more preferentially an isolate (solids more than 80% protein). Any method well known to the skilled person for this purpose may be used, whether by the dry process (extraction without use of aqueous solvent, e.g. via turbo-separation) or by the wet process (obtaining a flour by grinding, suspending the flour in an aqueous solvent, extracting the insoluble compounds, in particular the starch and the internal fibers, isoelectrically precipitating the proteins belonging to the groups of the globulins, recovering the latter by centrifugation). Pea proteins obtained by the wet process are particularly preferred since this process leads to obtaining an isolate that concentrates as much as possible the capacity thereof to activate the synthesis of FGF19.

The pea proteins (flour, preferentially concentrate, even more preferentially isolate) are then hydrolyzed. Any protein hydrolysis method, whether chemical, physical, enzymatic or biological, well known to a skilled person can be used in order to obtain the legume proteins, preferentially of pea, to be used for the invention. Enzymatic hydrolysis is preferred, in particular a hydrolysis carried out with protease belonging to the subgroup of the aminopeptidases coming from Aspergillus oryzae.

The preparation of the pea protein hydrolysate suitable for the invention therefore preferentially comprises enzymatic or non-enzymatic hydrolysis, so that said pea protein isolate has a degree of hydrolysis (DH) of between 5% and 10%, preferably between 6% and 8%, even more specifically from 6.5% to 7%.

In a first embodiment, the hydrolysis is carried out by an endopeptidase. A non-specific endopeptidase, derived from a strain of Aspergillus in particular a strain of Aspergillus spp. or Aspergillus oryzae, is selected. More particularly, an EC 3-4-11 endopeptidase is selected.

The exact amount of enzyme added to the suspension to obtain the desired characteristics of the pea protein isolates varies depending on specific characteristics such as the enzyme or the enzymatic system used; the desired final degree of hydrolysis; and/or the desired final molecular weight distribution. Since these parameters are known, a skilled person can easily determine the appropriate conditions for obtaining the desired characteristics of the pea protein isolate.

In one particular embodiment, the initial pea proteins used to prepare the pea protein isolate according to the invention are a pea protein composition as described in application WO 2007/17572 or prepared by a method as described in application WO 2007/17572 (the teaching being incorporated by reference). In one particular embodiment, the initial pea protein composition is the composition sold by ROQUETTE FRERES under the tradename NUTRALYS® S85F.

In a preferred embodiment of the invention, the pea protein suspension is brought to a value of 5 to 20% by weight of solids, in particular 15 to 20%. The reaction temperature is adjusted to a value of between 50 and 60° C., preferably of the order of 55° C. As a general rule, the enzymatic system or an enzyme is added to the suspension in amounts in the range from about 0.3 to 1% weight/volume. The hydrolysis reaction is typically carried out for a desired time in order to obtain the desired degree of hydrolysis and/or the desired molecular weight profile, in this case for a period of about 45 minutes to about 2:30 hours, preferably about 1 hour. Once again, the time required for the hydrolysis reaction depends on the characteristics as noted above, but can be easily determined by a skilled person.

In other embodiments, the suspension containing pea proteins can be hydrolyzed using non-enzymatic means, for example by mechanical (physical) and/or chemical hydrolysis. This technique is also well known in the state of the art.

Once the pea proteins have been hydrolyzed to the desired degree, the hydrolysis reaction is stopped, for example, by inactivating the enzyme, or by other conventional means. In one embodiment, the inactivation of the enzyme is carried out by heat treatment. In accordance with established practice, the enzyme preparation can be suitably inactivated by increasing the temperature of the incubation suspension to a temperature at which the enzymes become inactivated, for example to about 70° C. for about 10 minutes. The pea protein isolates thus obtained are then treated at a high temperature for a short time (HTST), then pasteurized and optionally concentrated to a solids content of 10 to 30% before being dried by atomization. For example, the isolate can be pasteurized at a temperature of between 130° C. and 150° C. for a period of about 1 second to about 30 seconds.

In a second preferential alternative embodiment of the invention, the legume proteins are faba bean proteins, the degree of hydrolysis of which is not modified by hydrolysis after its extraction, preferentially with a degree of hydrolysis of between 0% and 5%.

As will be demonstrated hereinafter in the section relating to the examples of this patent application, it is preferable within the meaning of the invention to use faba bean proteins over pea proteins. Indeed, the effect of activating synthesis is greater in particular in a context of cellular cooperation. This finding of greater efficacy was demonstrated using faba bean proteins, the degree of hydrolysis of which has not been increased with enzymatic, physical or biological hydrolysis.

The faba bean protein is obtained by any means well known to the skilled person, whether by the dry process (extraction without use of aqueous solvent, e.g. via turbo-separation) or by the wet process (obtaining a flour by grinding, suspending the flour in an aqueous solvent, extracting the insoluble compounds, in particular the starch and the internal fibers, isoelectrically precipitating the proteins belonging to the group of the globulins, recovering the latter by centrifugation). Faba proteins obtained by the wet process are particularly preferred since this process leads to obtaining an isolate that thus concentrates as much as possible the capacity thereof to activate the synthesis of FGF19.

A particularly preferred method for obtaining faba bean proteins is as follows:

The first step consists of using faba bean seeds. These seeds still comprise their protective external fibers, also referred to as hulls. The seeds may undergo a pre-treatment which can comprise steps of cleaning, sieving (for example, for separating seeds and stones), soaking, bleaching, toasting.

Preferably, if bleaching is performed, the heat treatment scale will be 3 minutes at 80° C. Nonlimiting examples of varieties are Tiffany, FFS or YYY. Preferentially, field bean seed varieties with a naturally low tannin and/or polyphenol content, such as the Organdi variety, will be used. Such varieties are known and can be obtained by varietal crossing and/or genetic modification.

The second step relates to the most effective possible separation of the external fibers and the cotyledons. It begins, for example, with a first grinding of the faba bean seeds using a stone mill. A specific, particularly appropriate example of such a stone mill is, for example, marketed by the company Alma®. As previously disclosed, the seed is inserted into a space formed by two stone discs, one of which is rotating. The applicant has noticed that this technique is particularly interesting since it produces a highly effective separation of the external fibers and the cotyledons of the seeds. Preferably, the inter-disc space is adjusted between 0.4 and 0.6 mm.

The ground material is then subjected to a counter-current ascending air flow. The various solid particles are classified according to their density. Typically, after equilibrium, two fractions are obtained: a light fraction containing mostly the external fibers or hulls and a “heavy” fraction containing mostly the cotyledons. A specific, particularly appropriate example of an adequate apparatus is for example the MZMZ 1-40 marketed by the company Hosokawa-alpine®.

The heavy fraction, enriched in cotyledons, is then ground using a knife mill. A specific, particularly appropriate example of such a knife mill is for example the SM300 marketed by the company Retsch®.

The succession of the three operations cited hereinbefore in the second step aims separate to very finely the external fibers and the cotyledons, avoiding damaging these two parts and mixing them.

The third step aims to reduce the particle size of the heavy fraction enriched with cotyledons by grinding same using a roller mill. A specific, particularly appropriate example of such a roller mill is, for example, the MLU 202 marketed by the company Buhler®. It is used herein in order to reduce the overall particle size of the flour, in order to obtain a uniform, sufficiently fine powder so as to facilitate the following step 4. The preferred particle size is comprised between 200 and 400 microns, preferentially 300 microns. In order to measure this particle size, a laser particle size analyzer is preferably used, although any method is possible, such as sieving.

Alternatively, the step of reducing the particle size of the heavy fraction enriched with cotyledons can be carried out in the presence of aqueous solvent, preferentially water. In this case, the fourth step below is merged with the third step which are then performed concomitantly.

The fourth step aims to place the powder obtained in the preceding third step in suspension in an aqueous solvent, preferentially in water. The aim here is to perform a selective extraction of certain components, mostly the proteins as well as the salts and the sugars, by solubilizing them. The pH of the solution is advantageously rectified towards a neutral pH in order to limit the solubilization of tannins and polyphenols as much as possible. This pH rectification can be carried out before and/or after suspending the powder in the aqueous solvent.

The aqueous solvent is preferentially water. The latter may, nevertheless, contain additives, for example with compounds that make it possible to facilitate the solubilization. The pH of the aqueous solvent is adjusted between 6 and 8, preferentially 7. Any acidic or basic reagent such as soda, lime, citric or hydrochloric acid is possible, but potash and ascorbic acid are preferred. The temperature is adjusted between 2° C. and 30° C., preferentially between 10° C. and 30° C., preferentially between 15° C. and 25° C., even more preferentially to 20° C. This temperature is controlled throughout the entire extraction reaction.

The powder obtained is diluted in order to obtain a suspension comprised between 5% and 25%, preferentially between 5% and 15%, preferentially between 7% and 13%, even more preferentially between 9% and 11%, the most preferred being 10%, the percentage being expressed as a percentage of powder by total weight of the water/powder suspension. The suspension is stirred using any apparatus known to a skilled person, for example a vat provided with a stirrer, provided with blades, marine propellers or any equipment that allows effective stirring. The extraction time, preferentially while stirring, is comprised between 5 and 25 minutes, preferentially between 10 and 20 minutes, even more preferentially 15 minutes.

The fifth step aims to separate by centrifugation the soluble fraction and the solid fraction obtained during the fourth step. The preferred industrial principle can be found in patent application EP1400537, which is incorporated herein by reference. The principle of this method is to start by using a hydrocyclone in order to extract a fraction enriched in starch, then to use a horizontal decanter in order to extract a fraction enriched in internal fibers. Nevertheless, it is possible to use an industrial centrifuge which extracts a fraction enriched with starch and internal fibers. In every case, solid fractions and a liquid fraction that concentrates most of the proteins are obtained.

The sixth step aims to acidify to the isoelectric pH of the field bean proteins, around 4.5, and then to subject the solution to heating in order to coagulate the proteins referred to as globulins, which are separated by centrifugation.

The acidification is carried out to a pH between 4 and 5, preferentially 4.5. This is preferentially done with hydrochloric acid at about 7% by weight, but all types of acids, mineral or organic, can be used such as citric acid. Even more preferentially, the use of pure ascorbic acid or ascorbic acid in combination with another mineral or organic acid, is also possible. The use of ascorbic acid to acidify helps improve the final coloring. Any heating means is then possible, for example by means of a stirred vat provided with a double shell and/or coil or an in-line steam-injection cooker (“jet cooker”). The heating temperature is advantageously between 45° C. and 75° C., preferentially between 50° C. and 70° C., even more preferentially between 55° C. and 65° C., the most preferred being 60° C. The heating time is between 5 minutes and 25 minutes, preferentially between 10 and 20 minutes, the most preferred being 10 minutes.

The protein composition, mostly globulin, coagulates and precipitates within the solution. It is separated by any centrifugation technique, for example such as the Flottwegg® Sedicanter. The residual solution obtained concentrates sugars, salts and albumins, it is referred to as field bean solubles. It is processed separately, preferentially evaporated and/or dried.

The combination of isoelectric precipitation with controlled heating proposed by the invention makes it possible to obtain:
a floc of coagulated proteins, resulting after the required treatment in the product claimed in the present application, and
residual solubles containing among others soluble proteins (albumins), salts and sugars

In a seventh step, the protein composition is then diluted to around 15-20% by weight of solids and neutralized to a pH between 5.5 and 6.5, preferentially 6.5, by means of any basic agent, preferentially potash at 20% by weight.

The protein composition can then undergo a thermal treatment, preferentially at a temperature of 135° C. by direct steam injection through a nozzle and flash vacuum cooling to 65° C.

The protein composition obtained can be used directly for example by being hydrolyzed by a protease or else texturized by an extruder.

In an eighth step, the protein composition useful for the invention is dried. The preferred drying mode is atomization, in particular using a multiple-effect atomizer. The typical parameters are an input temperature of 200° C. and a vapor temperature of 85-90° C.

### Therapeutic Use

The composition according to the present disclosure makes it possible to activate the synthesis of FGF19 in human cells.

FGF19 is a gene coding for a protein belonging to the family of the fibroblast growth factors, which govern nutrient metabolism. In humans, the FGF19 gene (Gene ID: 9965 updated on Jul. 8, 2021) codes for a transcript of 1821 bp (NCBI Reference Sequence: NM_005117.3, updated on Jun. 29, 2021) which codes for an FGF19 protein (NCBI Reference Sequence: NP_0051108.1, updated on Jun. 29, 2021). FGF19, also referred to as FGF15 in rodents, is a member of a subfamily of fibroblast growth factors that govern nutrient metabolism. FGF19 is expressed and secreted in the distal small intestine, by the bile and intestinal epithelial cells, wherein its synthesis is positively regulated after the postprandial absorption of bile acids.

The term “activation of the synthesis” or “overexpression of the gene” should be understood within the meaning of the invention to be an exogenous activation (that is to say, by means of a compound, of a foreign molecule) of the synthesis of mRNA or of protein in the cells of a living organism, in particular the synthesis of FGF19 in the context of this invention.

Protein synthesis is the set of biochemical processes that allow the cells to produce their proteins from their genes. It covers the steps of transcribing the DNA into messenger RNA, of aminoacylating the transfer RNAs, of translating the messenger RNA into polypeptide chains, of post-translationally modifying the latter, and finally of folding the proteins thus produced. It is closely regulated at multiple levels, mainly during the transcription and the translation. For human beings, the gene coding for the expression of FGF19 is located on human chromosome 11.

The activation of the synthesis of FGF19 can be determined by measuring the level of expression of the FGF19 gene, in particular by measuring either the level of expression of FGF19 mRNA, or the level of expression of the FGF19 protein by any methods known to a skilled person. In particular, the expression of FGF19 is increased when the level of expression is at least 1.5 or 2, 3, 4 or 5 times greater than in the untreated cells.

The term “FGF19 protein” or “FGF19 polypeptide” should be understood within the meaning of the invention as a polypeptide, that is to say an amino acid chain, which is homologous with the particular amino acid sequence naturally secreted in the bodies of mammals.

According to the invention, the terms “FGF19 polypeptide”, “FGF19” and “FGF15/19” refer to the native sequence of a natural form of an FGF19 polypeptide as expressed in the body of any mammal. This term comprises any isoform of natural origin, which encompasses alternative forms such as alternately spliced forms, allelic variant forms, and the untreated and treated forms of FGF19, such as the forms of FGF19 polypeptide comprising a signal peptide

This term “FGF19 polypeptide” also comprises fragments of a natural form of an FGF19 polypeptide, in particular recombinant fragments having the same biological activity as said natural form

In the meaning of the invention, the term “FGF19” comprises all the FGF19 polypeptides being at least 50% identical to the human sequence represented in SEQ ID NO: 1. The expression “an FGF19 polypeptide being at least 50% identical to the human sequence represented in SEQ ID NO: 1” refers to a polypeptide, a member of the FGF19 family, having an amino acid sequence being at least 50% amino acid identical to the reference sequence. This requires, after alignment, for 50% of the amino acids in the candidate sequence to be identical to the corresponding amino acids in the reference sequence

The term “amino acid identical” means that the same amino acid is observed in both sequences. The identical nature does not take into account post-translational modifications that may occur on the amino acids. The identical nature according to the present invention is determined by means of a computer analysis, such as the ClustaIW computer alignment program, and the default parameters suggested therein. The ClustaIW software is available from the website http://www.clust.org/clust2/. Using this program with its default settings, the part of a request and a “reference polypeptide” are aligned. The number of totally conserved residues is counted and divided by the length of the reference polypeptide. According to the present invention, the “reference polypeptide” has the sequence as shown in SEQ ID NO: 1

The expression “at least 50% identical” indicates that the percentage to which the two sequences, the request and the reference polypeptide of SEQ ID NO: 1, are identical is at least 50, 55, 60, 65, 70, 75, 80, 85, 86, 87, 88, 89, 90, 91, 92, 93, 94, 95, 96, 97, 98, 99, or 100% with respect to the sequence SEQ ID NO: 1.

Therefore, the FGF19 polypeptide is selected from the FGF15 polypeptide expressed in mice, the FGF19 polypeptide expressed in humans, or the homologues of FGF15 and FGF19 expressed in other mammals such as rats, dogs, cats, sheep, cattle, horses, pigs, goats, rabbits.

The members of the FGF19 family comprise, especially:
the human FGF19 polypeptide of 216 amino acids (including 24 amino acids constituting the signal peptide), the sequence of which is represented in SEQ ID NO: 1;
the Mus musculus FGF15 polypeptide of 218 amino acids (including 25 amino acids constituting the signal peptide), the sequence of which is represented in SEQ ID NO: 10.
any one of the other sequences as represented in SEQ ID NO: 2, 3, 4, 5, 6, 7, 8, or 9, as presented in table 1 hereunder:

TABLE 1

| | | Number | Homology with |
|---|---|---|---|
| Sequence | Mammal | of amino | SEQ ID NO: |
| number | species | acids | 1 (in %) |
| SEQ ID NO: 1 | Homo sapiens | 216 | 100% |
| SEQ ID NO: 2 | Sus scrofa | 220 | 75.93% |
| SEQ ID NO: 3 | Bos taurus | 218 | 73.95% |
| SEQ ID NO: 4 | Equus equalicule | 94 | 90.43% |
| SEQ ID NO: 5 | Ovis | 137 | 73.72% |
| SEQ ID NO: 6 | Canis familiale family | 193 | 80.31% |
| SEQ ID NO: 7 | Felis catus | 219 | 81.94% |
| SEQ ID NO: 8 | Oryctolagus | 219 | 72.56% |
| | cuniculus | | |
| SEQ ID NO: 9 | Rattus norvegicus | 218 | 53.11% |
| SEQ ID NO: 10 | Mus musculus | 218 | 52.63% |

The present invention consists more particularly of the therapeutic use of a composition of legume proteins, preferentially of pea or faba beans, the degree of hydrolysis of which is less than 10%, in order to activate the synthesis of FGF19 in a subject and to prevent or treat diseases susceptible to treatment by overexpression of FGF19, especially diseases associated with the dysregulation of FGF19.

Subjects suffering from diseases associated with the dysregulation of FGF19 have a dysregulation of the expression of the FGF19 gene, in particular a reduction in the synthesis of FGF19 or a decrease in the FGF19 signaling pathway, especially a decrease in the activation of the factors of the downstream signaling pathway of FGF19 such as ERK, PI3K, GSK3β.

In one particular embodiment, the composition as described previously is used for the treatment and/or prevention of a disease, preferably a disease susceptible to treatment by activation of the synthesis of FGF19, especially a disease associated with dysregulation of the FGF19 signaling pathway, preferably sarcopenia or a metabolic disease such as a chronic inflammatory bowel disease, primary bile acid malabsorption, obesity or non-alcoholic fatty liver disease (NAFLD), such as non-alcoholic steatohepatitis (NASH) in a subject.

The term “subject” or “patient” should be understood within the meaning of the invention as any living being belonging to the animal kingdom. In a non-exhaustive manner, this therefore includes dogs, cats, cows, pigs, etc. Preferably, “subject” is intended to mean a human being, even more preferably a human being having a disease or syndrome susceptible to treatment by the activation of the synthesis of FGF19.

Preferably, the method according to the invention is characterized in that the subject is a human being, in particular an elderly person. “Elderly person” means a human being who is older than 40, 45, 50, 55, 60, 65, 70, 75, 80, 85, 90, 95, 100, or 105 years old.

Preferably, the subject is an animal, in particular selected from the list of cats, dogs, cows, sheep, pigs, chickens, and turkeys

The term “treatment” includes a curative treatment resulting in a healing or a treatment that alleviates, improves, eliminates, reduces, and/or stabilizes the symptoms of a disease or the suffering that it causes. The term “prevention” corresponds to a prophylactic or preventive treatment, which comprises a treatment that results in the prevention of a disease as well as a treatment that reduces and/or delays the incidence of a disease or the risk of it occurring.

In a first preferred embodiment, the composition according to the invention aims to increase the anabolism of muscle fibers via the activation of the synthesis of FGF19 and to prevent or treat diseases associated with muscle loss, in particular sarcopenia. Sarcopenia is a geriatric syndrome, which is characterized by a reduction in muscle capacities due to age and which, as it worsens, causes a deterioration of muscle strength and physical performance.

The composition according to the invention preferably makes it possible to prevent or treat sarcopenia and to improve or eradicate the disease or the symptoms associated with the disease, such as the deterioration of muscle strength and physical performance.

The consumption of legume protein is already well known, but for this purpose it is clearly recommended to combine it with other protein sources in order to improve the amino acid profile. The innovative preferential strategy discovered by the applicant simplifies things by concentrating on the specific activity on the overexpression of FGF19. This overexpression is maximal for the legume proteins, preferentially of pea and or faba bean, when the degree of hydrolysis is very precisely selected. This approach is therefore completely opposite to the strategies commonly used in the background art.

In another preferred embodiment, the composition according to the invention makes it possible to prevent or treat a metabolic disease via the activation of the synthesis of FGF19, in particular non-alcoholic steatohepatitis (or NASH).

NASH is liver damage resulting from excessive lipid accumulation in the hepatocytes, leading to lipotoxicity and inflammatory lesions of the hepatocytes. The physiopathology involves the accumulation of fat (steatosis), inflammation and, in a variable manner, fibrosis.

The composition according to the invention makes it possible to prevent or treat NASH and to improve or eradicate the disease or the symptoms associated with NASH, such as a decrease in lipid accumulation in the hepatocytes and inflammatory lesions of the patient's hepatocytes.

To date, few or no treatments exist. A reduced and balanced diet, exercise, drinking coffee or taking vitamin E are, for now, the only treatments advised by the “American Liver Foundation” (https://liverfoundation.org/for-patients/about-the-liver/diseases-of-the-liver/nonalcoholic-steatohepatitis-information-center/nash-treatment/). These recommendations are unfortunately meager, and sometimes difficult to apply (physical exercise not possible, caffeine consumption). The composition according to the invention therefore makes it possible to consider, via the stimulation of FGF19 synthesis, a route for combatting the prevalence of this NASH.

The present invention also consists of the use of a composition comprising a legume protein as described previously for the preparation of a drug to be used in the treatment of a disease susceptible to treatment by the activation of the synthesis of FGF19 as described previously, preferably sarcopenia or NASH.

In another particular embodiment, the present disclosure is a method for treating a disease susceptible to treatment by the activation of the synthesis of FGF19 as described previously, preferably sarcopenia or NASH, in a subject in need thereof, which comprises administering to a subject a therapeutically effective amount of a composition comprising a legume protein as described previously.

In the context of the disclosure, a therapeutically effective amount refers to a dose that is high enough to reverse, relieve or inhibit the progression of the disorder or the state to which this term applies, or to reverse, relieve or inhibit the progression of one or more symptoms of the disorder or the state to which this term applies.

The effective dose is determined and adjusted depending on factors such as the composition used, the route of administration, the physical characteristics of the individual under consideration, such as their sex, age and weight, the concomitant medication, and other factors, that persons competent in the medical field will recognize.

According to one particular embodiment of the invention, the composition further comprises at least one FXR (Farnesoid X Receptor) agonist. FXR agonists are well known to a skilled person and can be selected by way of non-limiting examples from: obeticholic acid (OCA), Chenodeoxycholic acid (CDCA), 6a-ethyl-chenodeoxycholic acid (6-ECDCA), Alisol B 23-acetate (AB23A), Cafestol, Fexaramine, GW4064 (3-(2,6-Dichlorophenyl)-4-(3′-carboxy-2-chlorostilben-4-yl)oxymethyl-5-isopropylisoxazole), and Tropifexor, preferably GW4064 and Chenodeoxycholic acid (CDCA).

The composition according to the present disclosure may be included in a pharmaceutical composition, optionally in combination with a pharmaceutically acceptable excipient.

The present invention thus consists of a pharmaceutical composition intended to activate the synthesis of FGF19 comprising legume proteins, preferentially selected from pea or faba bean, the degree of hydrolysis of which is less than 10% as described previously, optionally in combination with a pharmaceutically acceptable excipient.

The first essential constituents for the pharmaceutical composition intended to activate the synthesis of FGF19 are legume proteins, preferentially of pea or faba bean, the degree of hydrolysis of which is less than 10%, as described in the preceding paragraphs. In particular, the two preferential alternative embodiments respectively use a pea protein, degree of hydrolysis of which is preferentially between 5% and 10%, or a faba bean protein, degree of hydrolysis of which is not modified by hydrolysis after its extraction, preferentially with a degree of hydrolysis of between 0% and 10%, preferentially between 0% and 5%.

In a preferred embodiment, these proteins constitute the only protein sources of the composition according to the invention. Unlike the solutions that are well known in the background art, the composition does not use a combination with other protein sources, for example rice or wheat proteins. The composition in this preferential embodiment aims to maximize the capacity of the legume proteins to activate the synthesis of FGF19. Amino acids can be added to improve the profile of the composition, although this is not necessary.

The term “pharmaceutical composition” should be understood to be any composition intended to treat a subject. Non-limiting examples of such pharmaceutical compositions are given in the rest of this present application.

A “pharmaceutically acceptable excipient” refers to a carrier which does not produce an undesirable reaction, allergic reaction or other unpleasant reaction when it is administered to a mammal, in particular a human, according to the case. A pharmaceutically acceptable carrier or excipient refers to a solid, semi-solid or liquid non-toxic filler, a diluent, an encapsulation material or a formulation aid of any type.

Preferably, the pharmaceutical composition contains carriers, which are pharmaceutically acceptable for a formulation capable of being orally administered.

The composition may be any composition well known to a skilled person such as, for example (and in a non-limiting manner), ready-to-drink (RTD) beverages, powders to be reconstituted (powder mixes), compotes, gels, breadmaking products, dairy products, . . . .

More particularly, the invention relates to the application of these formulations to beverages, via mixtures of powders to be reconstituted, for diet nutrition (sport, slimming), and to ready-to-drink beverages for clinical (oral route or enteral bag) and dietary nutrition, wherein a low viscosity of the beverage and an improvement in the solubility of the legume protein are sought

Likewise, the invention relates to the application of these nutritional formulations in dairy or plant beverages, in yogurt-type fermented milks (blended, Greek, drinkable) and in dairy or plant creams, frozen desserts or sorbets.

Finally, the invention relates to the application of these nutritional formulations in biscuits, muffins, pancakes, nutritional bars (intended for specialized/slimming or sports nutrition), in protein-rich bread or gluten-free bread, in high-protein small cereals obtained by cooking-extrusion (“crisps”), wherein high-protein solutions are more particularly sought that do not have a negative impact on the preparation process or the texture of the finished preparations or products.

Within the meaning of the invention, “powdered nutritional formulations” refers to powdered formulations comprising at least, preferentially only, a legume protein, and in particular of pea or faba bean, according to the invention, which can be reconstituted with an aqueous liquid, and which are suitable for oral administration to a human being.

The expression “dry mixture” as used herein, unless specified otherwise, refers to the mixing of the components or ingredients to form a base nutritional powder or, to the addition of a dry, powder or granulated component or a powder-based ingredient to form a powdered nutritional formulation.

All the percentages, parts and ratios, as used herein, relate to the weight of the total formulation, unless specified otherwise.

The powdered oral formulations of the present invention are generally in the form of flowable or substantially fluid particulate compositions, or at least particulate compositions that can be easily molded and measured using a spoon or another similar device, wherein the compositions can easily be reconstituted by the intended user with an aqueous solution, typically water, in order to form a liquid formulation for immediate oral or enteral use. In this context, use “immediately” generally means for about 48 hours, more typically for about 24 hours, preferably right after reconstitution.

The powdered formulations can be formulated with all types and amounts of sufficient nutrients so as to form a dietary supplement, or a specialized nutritional formulation intended to be used in persons following a particular diet intended for sports and slimming.

In exemplary embodiments, the powdered nutritional formulation may be formulated to be used:
for the diet of an overweight person following a weight-loss diet and seeking to minimize the loss of muscle mass,
for repairing the muscles after an intense effort, for example in athletes, for enabling athletes to maintain or build muscle mass, or
as a meal substitute for people seeking to lose weight via a satiogenic effect.

The powdered formulations may have a caloric density suited to the nutritional needs of the end user, although in most cases the reconstituted powders comprise from about 350 kcal to about 400 kcal per 100 ml.

The powdered formulations may have a protein content suited to the nutritional needs of the end user, although in most cases the reconstituted powders comprise from about 20 g to about 91 g of protein per 100 g, including from about 40 g to about 65 g of proteins per 100 g.

Thus, the formulation may comprise between 20 and 95% of proteins with respect to the total weight of the formulation, for example 20-90%, 30-80%, or 40-60%.

For example, the legume protein isolate, preferentially of pea or faba bean, according to the present invention may represent 40-50%, 50-60%, 60-70%, 70-80%, 80-90%, or 90-100% of the total protein content of the formulation, or any combination of these percentage ranges. 100% being the ultimate preferential embodiment in order to maximize the effect of FGF19 overexpression

Furthermore, the powdered food formulations may have a fat content suited to the nutritional needs of the end user, although in most cases the reconstituted powders comprise from about 0.5 g to about 13 g per 100 g, including from about 3 g to about 7 g per 100 g. Thus, the formulation may comprise between 0 and 20% of lipids with respect to the total weight of the formulation, for example 0.5-15%, 1-10%, or 3-7% (in particular % by weight).

The powdered nutritional formulations of the present invention can be packaged and sealed in single or multi-use containers, and then stored under ambient conditions for up to about 36 months or longer, more typically from about 12 to about 24 months.

For multiple uses of the containers, they may be opened and closed for repeated use by the user, provided that the closed packet is then stored under ambient conditions (for example, avoiding extreme temperatures) and the contents are used in about one month or two.

The fields of application of the nutritional formulations according to the invention are, especially:
diet nutrition (sport, slimming),
clinical nutrition (in the form of a beverage, a dessert cream or an enteral bag),
dairy products (in the form of yogurts, dairy beverages, dairy creams, frozen desserts or sorbets).
biscuit products, pastry products, breadmaking products and high-protein cereal products.

In the field of sports, it is known that proteins play a role in muscle maintenance and growth. Protein intake is also important for athletes who practice weight lifting or resistance training.

Ready-to-drink protein-rich or high-protein beverages thus provide the body with an intake of selected proteins, with fewer calories.

These high-protein beverages must:
be rich in proteins, low in carbohydrates and fats;
have a good taste;
be designed to assist with weight loss, stimulating fat burning and helping with muscle recovery;
be satiogenic;
be useful for managing cravings, without any added sugars or fats;-present balanced content of essential amino acids, fiber, vitamins and minerals;
be low-calorie.

These ready-to-drink beverages can advantageously be prepared with the isolates of legume proteins, preferentially of faba beans or pea, in accordance with the invention. Moreover, they can be used as the sole protein source, preferably since they maximize the effect of overexpression of FGF19.

For example, plant beverages that provide an alternative to cow's milk contain, on average, 4.5 g to 11 g of proteins per 100 ml of beverage, preferably of the order of 7 g of proteins per 100 ml, and are very low in fiber (of the order of 0.5 g to 1 g per 100 ml).

Thus, the beverage may comprise between 1 and 20% of proteins with respect to the total weight of the beverage, for example 3-15%, or 6-8%.

For example, the pea protein isolate according to the present invention may represent 50-60%, 60-70%, 70-80%, 80-90%, or 90-100% of the total protein, or any combination of these percentage ranges. Preferably, it represents at least 52%. Especially, the intake of pea proteins is between 52 and 100% of the total protein intake.

For ready-to-drink beverages, the intake of pea proteins can range from 0 to 100%, preferably from 0.01 or 0.1% to 100%. For example, the pea protein isolate according to the present invention may represent 0.1-10%, 10-20%, 20-30%, 40-50%, 50-60%, 60-70%, 70-80%, 80-90%, or 90-100% of the total protein, or any combination of these percentage ranges.

In the field of “slimming” beverages, i.e. those intended to be used in calorie-restricted diets or weight-loss diets, as mentioned above, these high-protein or protein-enriched beverages are not only effective for rapid muscle gain. This type of beverage is also highly advantageous in the context of a slimming diet based on protein consumption.

It is known that slimming beverages are ideal for assisting in weight loss. More particularly, they make it possible:
to provide a satiating effect
to protect the muscles and tone the body, avoiding weight gain.

Like “sports” beverages, these slimming beverages have:
a balanced content of essential amino acids, fiber, vitamins and minerals
low contents of sugars, fat and calories.

In this way, protein-rich beverages are in fact highly effective for quickly losing a few kilos. These protein-rich preparations simply reduce or stop the sensation of hunger in the person who consumes them. By drinking such a beverage, for example, a user can considerably reduce the amount of food they eat, and enable rapid weight loss (in the context of a meal substitute process for weight control, or as a substitute for total daily food intake for weight control).

In clinical nutrition, it is known that enteral nutrition is a therapeutic solution for feeding by tube that is used when the digestive tract is functional and accessible but when the patient cannot normally eat or in cases of severe malnutrition.

This technique makes it possible to supply nutrients directly into the digestive tract. It replaces, totally or partially, conventional oral feeding with “complete” nutrient formulas that provide all of the nutrients that the body requires.

These formulas are generally packaged in flexible bags (made of PVC) and administered by means of nasogastric tubes, gastrostomies, nasojejunal tubes, nasoduodenal tubes, jejunostomy.

These nutritional mixtures consist of proteins, lipids, carbohydrates, vitamins and minerals, with or without fiber.

Several categories are distinguished: polymeric mixtures (standard) and semi-elementary mixtures (“predigested”), the latter being indicated in very specific cases (short bowel syndrome, exocrine pancreatic insufficiency, etc.):
Polymeric mixtures:
hypocaloric (0.5-0.75 kcal/ml), normal-or high-protein, with or without fiber
isocaloric (1 kcal/ml), normal-or high-protein, with or without fiber
hypercaloric (1.25-1.5 kcal/ml), normal-or high-protein, with or without fiber
specific formulas (glycemic metabolism disorders, respiratory failure).

The semi-elemental mixtures are iso- or hypercaloric, normal-or high-protein blends, based on medium-chain peptides and triglycerides.

Pea protein isolates, as a protein source, given their functional properties, are particularly well suited to this use.

Moreover, they make it possible to preserve the same properties as milk proteins, with a lower cost.

The invention will be better understood with the following examples, which are only intended for better understanding the latter. These do not have a limiting scope.

### EXAMPLES

### Example 1: Preparation of a Composition Comprising a Hydrolyzed Pea Protein, the Degree of Hydrolysis (DH) of Which is Between 5% and 10%

Pea protein (sold by the applicant company under the tradename NUTRALYS® S85F) is suspended at 15% w/w in 8500 liters of water preheated to 55° C.

After stirring for 3 h at 55° C., 0.5% (weight/weight) of FLAVORPRO 750 MDP endoprotease (from the company BIOCATALYST) is added and the enzymatic reaction is allowed to establish for 1 hour at 55° C. The reaction is then inhibited by heating the medium to 70° C. and this temperature is maintained for at least 10 minutes.

Finally, a UHT treatment (scale 140° C.-10 s) and drying by atomization are carried out.

The powder obtained is characterized by having a degree of hydrolysis of 7 and a solids content of 95%

### Example 2: Preparation of a Food Composition Comprising a Non-Hydrolyzed Faba Bean Protein

Faba bean seeds of the Tiffany variety are first treated using a stone mill (Alma®). The ground material is then processed by turbo-separation using a so-called “zig-zag” system (MZM 1-40, Hosokawa-alpine®) with an air speed of 4.0 m.s-1 (23 m3.h-1). A light fraction containing the external fibers and a heavy fraction containing the cotyledons are thus obtained. The heavy fraction is then processed using a knife mill (SM300, Retsch®) with a rotation speed of 700 RPM, the outlet of which is fitted with a 6 mm screen. The heavy fraction is then ground using a roller mill (MLU 202, Bühler®). At the end a flour is obtained in which the particle size is less than 300 μm.

This flour is placed in suspension at 10% by weight of solids in drinking water at 20° C. The pH is adjusted to 7 by adding potash. Homogenization is carried out during 15 minutes also at 20° C. The solution is then sent into a Flottweg Sedicanter decanter (bowl speed: 60% (around 3500 g), screw speed at 60% for a Vr=18.8, pipette for the supernatant (overflow) at 140 mm, supply at 1 m³/h) and the liquid supernatant containing the proteins is retrieved.

This supernatant is acidified to pH 4.5 by adding hydrochloric acid to around 7% by weight. It is heated to 60° C. by injecting steam into a double shell of the vat, where homogenization is carried out during 15 minutes. The Flottweg Sedicanter is used a second time (bowl speed at 60% (around 3500 g), screw speed at 10% for a Vr=3.5 up to 40% (Vr =12.6), pipette for the overflow at 140 mm at the start until 137, supply at 700 l/h) but this time in order to retrieve the sediment that contains the coagulated proteins.

The sediment is diluted to around 15-20% by weight of solids and neutralized to pH 6.5 by adding 20% potash. A thermal treatment is performed at 135° C. by means of a nozzle and flash vacuum cooling to 65° C. is carried out. The product is finally atomized (input temperature of 200° C. and vapor temperature of 85-90° C.)

### Example 3: Materials and Methods

In addition to the two samples described in examples 1 and 2 hereinbefore and referred to as RQ3 and RQ7, respectively, the proteins listed hereinafter were used by way of comparison:
RQ1: PRODIETF90 whey protein (INGREDIA)
RQ2: standard egg albumin
RQ4: Nutralys® W (wheat protein isolate)
RQ5: Nutralys® H85 (pea protein hydrolysate, the DH of which, measured according to the protocol described in the present application, is equal to 22)
RQ6: PROATEIN® (oat protein concentrate)
RQ8: Tubermine GP (potato protein isolate)
RQ9: Solulys® 095^{E} (corn steep)
RQ10: pea albumin obtained according to example 2 of patent WO 2018/197822
RQ11: total pea proteins (albumins+globulins) obtained according to examples 1 and 2 of patent WO 2019/068998, the degree of hydrolysis of which has not been modified

The samples are first hydrolyzed in vitro in order to simulate their digestion following their absorption by a subject. The protocol used is as follows:

The in vitro digestion model used simulates the digestion of foods by mimicking three different phases of digestion: the oral phase, the gastric phase and the intestinal phase. In order to simulate mastication and wetting by saliva, a hydration phase is first carried out. 6 g of proteins are solubilized in a buffer consisting of calcium chloride (CaCl2) with a final concentration of 2.6 mM, for one hour at 37° C. under stirring. The gastric phase is simulated with the addition of a buffer solution containing 157.8 mM hydrogen chloride (HCl), and 157.8 μM CaCl2. The reaction mixture is adjusted to pH 2 with a 4M hydrochloric acid (HCl) solution or a 4M sodium hydroxide (NaOH) solution. Then 0.6 g of porcine pepsin (P7000, Sigma) are added to the rest of the reaction mixture in order to initiate the digestion of the proteins. The reagents are stirred for 2 hours at 37° C. with regular pH adjustments over time (pH=2±0.5). Digestion continues with the intestinal phase, adding a solution containing 210 mL of phosphate buffered saline (PBS) buffer at pH 7.4, 558 μM CaCl2 and 20.9 mM NaOH. The pH of the reaction mixture is then adjusted to 6.8. Then 1.2 g of pancreatin of porcine origin (P7545, Sigma) is added to complete the hydrolysis of the proteins into small peptides. The mixture is incubated for 4 hours under stirring at 37° C. with pH adjustments over time (pH=6.8±0.5). The enzymes are subsequently inactivated by placing the mixture for 10 minutes in a water bath at 90° C. The digestates obtained are frozen and then lyophilized. All of the proteins digested in vitro then underwent a post-treatment described hereinafter:
Dissolving in a sterile PBS solution at a concentration of 100 mg/mL
Homogenizing for 1 hour at 40° C. under stirring (80 rpm)
Centrifuging for 15 min at 5000 g
Filtering on filters with a porosity of 0.45 microns

Aliquots referred to as protein digestates (or PD in the rest of this application) of 1 mL are prepared and stored at −20° C.

The following reagents are also used in sterile, filtered DMSO Hybri-Max™:
FXR receptor agonist GW4064,
chenodeoxycholic acid (CDCA),
dexamethasone (glucocorticoid receptor agonist)

For cell cultures, the HT29 (ECACC 91072201), HT29-MTX and Caco-2 lines derived from a human colorectal adenocarcinoma are used, conventionally used as cell lines representative of human enterocytes. The cells are systematically cultured in Dulbecco's Modified Eagle Medium (DMEM): 4.5 g/L of glucose supplemented with 1% of sodium pyruvate, 1% of penicillin-streptomycin and 10% of decomposed fetal-bovine serum (FBS) (Eurobio, Les Ulis, France). For the 12-well and 96-well plate experiments, the HT29 cells were seeded with a density of 7.5×10⁴ cells/cm² with a medium changed every 2 days and the cells were exposed to different experimental conditions 2 days after confluence with a depleted complete medium in FBS. For the 12-well plates (Costar®, Cambridge, MA), Caco-2 and HT29-MTX cells were seeded in a ratio of 1:1 with a density of 2.5×10⁴ cells/cm². The cells were cultured for 14 days in a complete medium to allow differentiation. The medium was changed in the two compartments every two days until exposure to the complete medium was depleted in the FBS. The integrity of the cellular monolayer was evaluated by measuring the transepithelial electrical resistance (TEER) using a Millicell-ERS (Millipore Corp., Billerica, MA). The cell culture was sourced from Dutscher (Issy-les-Moulineaux, France), unless specified otherwise.

The protocol for exposing these cell cultures to the various samples and agonists is as follows: the cells were treated for 6 h with (i) either 1 mg/mL PD (this concentration showed no toxic effect on the HT29 cells) or nuclear receptor agonists, and (ii) the combination of PD and nuclear receptor agonists. The various conditions of the carrier as PD were dissolved in PBS and the agonists were dissolved in DMSO. After exposure, the cell supernatants were stored at −80° C. for the measurements of the secretion of FGF19 proteins, and the cell monolayers were maintained at −80° C. until total extraction of the RNA.

The effect of this exposure is measured by quantifying the messenger RNA of the FGF19 using the following protocol: the expression of the target genes was evaluated by quantifying their mRNA level in the cells by RT-qPCR. The total RNA was isolated using the TRI-Reagent solution (Sigma Aldrich). First-strand complementary DNA was synthesized from 1 μg of total RNA using the TAKARA Prime ScriptMC RT reagent kit (TAKARA Bio, Saint-Germain-en-Laye, France). The qPCR tests were carried out using the SYBR® Premix Ex-Taq™ kit (TAKARA Bio) on Rotor-Gene™ 6000 (Corbet Research, Mortlake, Australia). The mRNA levels of the human TATA binding protein (TBP) were used to normalize the data.

### Example 4: Evaluation of Cell Viability

The toxicity of the PDs, previously preheated for 10 min to 90° C., was evaluated on the HT29 cells in 96-well plates after 6 hours of exposure, at 1 and 2 mg/mL, in order to define the highest concentration which does not cause either degradation or lysis of the cells. Under such conditions, no PD has displayed a toxic effect (as evaluated by the release of LDH) at 1 or 2 mg/mL except for PD no. 3 (RQ3) which induced the appearance of round cells predicting cell detachment at 2 mg/mL. It was therefore decided to select the dose of 1 mg/mL to continue the tests on the expression of FGF19.

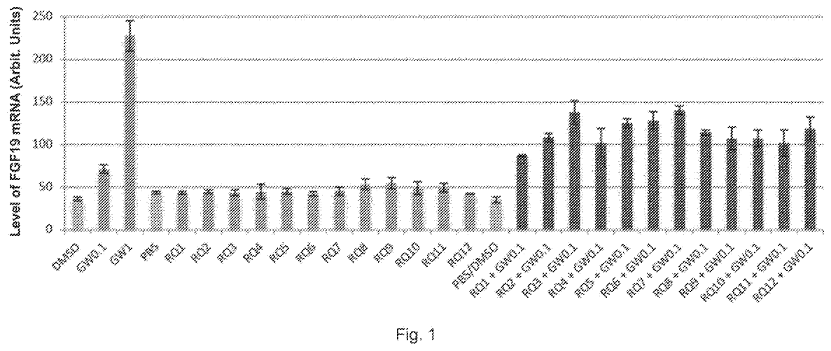

### Example 5: Effects of the Samples on the Expression of FGF19

[FIG. 1] summarizes the results obtained. The HT29 cells were exposed to the various PDs alone or in combination with an FXR agonist to determine their potential capacity to increase the expression of the FGF19 gene. As FXR agonist, GW4064 at the doses of 0.1 and 1 μM, increased the expression of the FGF19 gene in a dose-dependent manner by 2 and 6.2 times, respectively, compared to the control condition (carrier=DMSO). Exposure to the PD alone did not display any change in the expression of FGF19 compared to the control condition (carrier=PBS). However, the PD co-incubated with 0.1 μM GW4064 boosted the expression of FGF19 induced by the FXR agonist, with an increase of 2.5 (RQ1) to 4 times (RQ3 and RQ7) with respect to their control condition (PBS/DMSO) and an increase of 1.3 to 2 times with respect to the condition 0.1 μM GW4064.

It can be seen that RQ3 and RQ7 has the maximum capacity for overexpression of the gene coding for FGF19 with respect to the other proteins and in particular with respect to the proteins derived from the animal world such as RQ1 and RQ2.

It can also be noted that:
RQ3 is more effective than RQ5, which is also a pea protein isolate but with a degree of hydrolysis of 22, which is therefore greater than 10.
RQ3 is also more effective than RQ11 (non-hydrolyzed total pea protein)

These results clearly demonstrate that for the pea protein isolate according to the invention, the selection of a degree of hydrolysis of between 5% and 10% is therefore important in order to maximize the effect

### Example 6: Confirmation of the Effect

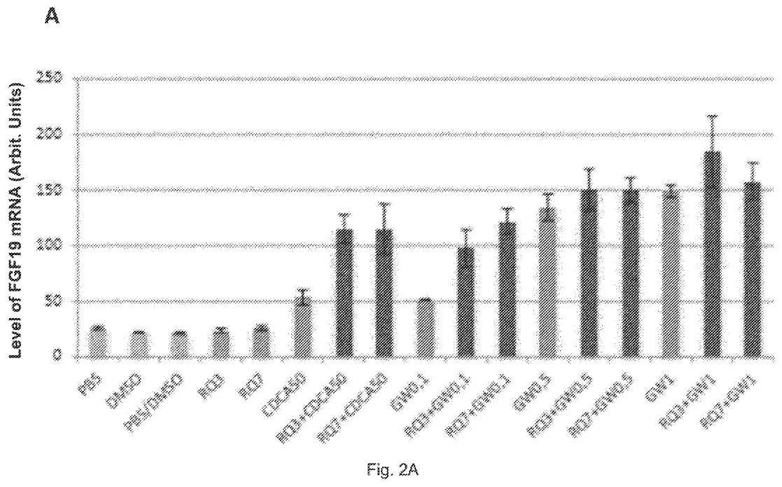

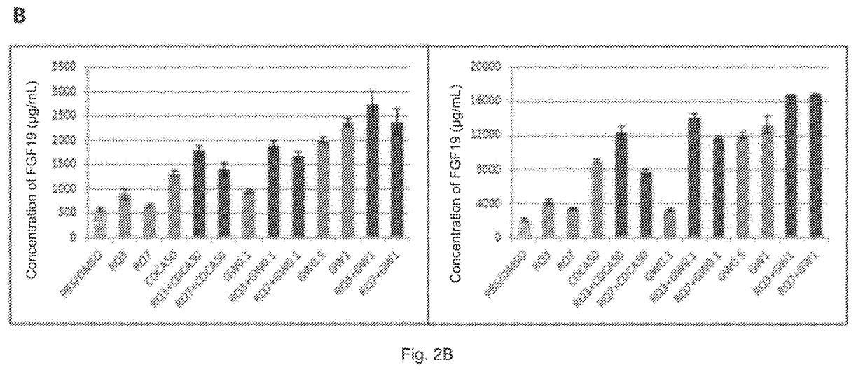

The capacity of the RQ3 and RQ7 digestates to increase the level of expression of the FGF19 mRNA and also the production and secretion of proteins was then evaluated. The HT29 cells were exposed to 0.1, 0.5 and 1 μM GW4064 and to a natural FXR agonist, 50 μM chenodeoxycholic acid (CDCA) alone or in combination with the RQ3 or RQ7 extracts. Confirming the first series of experiments, 0.1 to 1 μM GW4064 increased the expression of the FGF19 gene in a dose-dependent manner by 2.3 to 6.6 times, respectively, compared with the control condition (carrier=DMSO) [FIG. 2a]. The exposure to 50 μM CDCA showed a similar increase in the expression of the GW4064 gene at 0.1 μM (GW4064 therefore being more powerful than CDCA, in accordance with the data from the literature). RQ3 and RQ7 co-incubated with 50 μM CDCA both showed a 5.3 times increase with respect to the control condition (PBS/DMSO). The RQ3 digestate co-incubated with 0.1, 0.5 and 1 μM GW4064 induced a 4.5; 7 and 8.5 times increase of the expression of the FGF19 gene, respectively. The RQ7 digestate induced a 5.6; 7 and 7.3 times increase, respectively. The two digestates RQ3 and RQ7 co-incubated with FXR agonists boost the effect observed with the agonist alone on the expression of FGF19, with a greater effect for the low doses of agonists.

The level of expression of the protein is similar to that of the expression of the transcript, although the co-incubation of RQ3 and RQ7 with FXR agonists showed less boosting of the expression of the FGF19 protein than mRNA. The RQ3 digestate co-incubated with 50 μM CDCA and 0.1 and 1 μM GW4064 respectively caused a 3.1, 3.3 and 4.8 times increase in the level of FGF19 proteins with respect to the control condition (carrier) ([FIG. 2b], left). The RQ7 digestate, for its part, induces a 2.5, 3 and 4.2 times increase, respectively. As was observed in the expression of the genes, a greater boosting of the synthesis of FGF19 proteins occurs at the lowest dose of agonists, with almost no boosting induced by the PD with 1 μM GW4064 with respect to the agonist alone, which suggests that the maximum effect has been reached.

The same trends were observed when the cells were incubated for 24 h with the PDs ([FIG. 2b], right). RQ3 and RQ7 showed boosting of the production of FGF19 proteins, with a maximum effect with 0.1 μM GW4064.

### Example 7: Effect on a Co-Culture

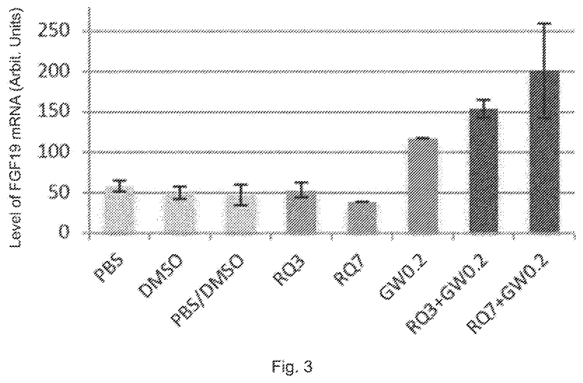

The capacity of RQ3 and RQ7 to induce the expression of FGF19 was evaluated in a co-culture model in an insert to resemble the physiological conditions of the intestines with apical and basolateral exchanges. Caco-2 cells make it possible to establish intercellular junctions providing a selectively permeable barrier, and the HT29-MTX cells have the ability to produce mucus at the top of the monolayer. This type of co-culture of the two cell lines therefore provides a model constituting the two cell types that are most represented in normal epithelium: enterocytes and caliciform cells. RQ3 and RQ7 showed a boosting of the expression of FGF19 when they were co-incubated with 0.2 μM GW4064 [FIG. 3], although this effect is less powerful than that observed previously in plate experiments containing only HT29 cells.

1. A legume protein composition, the degree of hydrolysis of which is less than 10%, preferably containing more than 90% by weight of globulins with respect to the total weight of the proteins, for use in the prevention and/or treatment of a disease susceptible to treatment by activation of the synthesis of FGF19 in a subject, wherein said disease is sarcopenia or non-alcoholic steatohepatitis.

2. The composition for use according to claim 1, wherein said legume proteins are the only protein source within the composition.

3. The composition for use according to claim 1, wherein the legume proteins are pea proteins, the degree of hydrolysis of which is preferentially between 6% and 8%.

4. The composition for use according to claim 1, wherein the legume proteins are faba bean proteins, the degree of hydrolysis of which is between 0% and 5%.

5. The composition for use according to claim 1, wherein the subject is a mammal, preferably a human being, preferentially a person who is more than 40 years old.

6. The composition for use according to claim 1, further comprising an FXR agonist selected from the following: obeticholic acid (OCA), Chenodeoxycholic acid (CDCA), 6α-ethyl-chenodeoxycholic acid (6-ECDCA), Alisol B 23-acetate (AB23A), Cafestol, Fexaramine, GW4064 (3-(2,6-Dichlorophenyl)-4-(3′-carboxy-2-chlorostilben-4-yl) oxymethyl-5-isopropylisoxazole), and Tropifexor, preferably GW4064 and Chenodeoxycholic acid (CDCA).

7. A pharmaceutical composition capable of overexpressing the synthesis of FGF19 comprising legume proteins, the degree of hydrolysis of which is less than 10%, and optionally an acceptable pharmaceutical excipient, preferably wherein said legume proteins are the only protein source within said composition.

8. The pharmaceutical composition according to claim 7, wherein the legume proteins are:
pea proteins, the degree of hydrolysis of which is preferentially between 6% and 8%; or,
faba bean proteins, the degree of hydrolysis of which is between 0% and 5%.

9. The pharmaceutical composition according to claim 7, further comprising an FXR agonist selected from the following: obeticholic acid (OCA), Chenodeoxycholic acid (CDCA), 6α-ethyl-chenodeoxycholic acid (6-ECDCA), Alisol B 23-acetate (AB23A), Cafestol, Fexaramine, GW4064 (3-(2,6-Dichlorophenyl)-4-(3′-carboxy-2-chlorostilben-4-yl) oxymethyl-5-isopropylisoxazole), and Tropifexor, preferably GW4064 and Chenodeoxycholic acid (CDCA).


SEQUENCE LISTING (10 sequences)

SEQ ID NO: 1 - protein, 216 aa, homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 2 - protein, 220 aa, Sus scrofa
AMRSAPSRCAVVRALVLAGLWLAAAGRPLAFSDAGPHVHYGWGESVRLRHLYTASPHGVSSCFLRIHSDGPVDCAPGQSAHSLMEIRAVALSTVAIKGERSGRYLCMGADGKMQGQTQYSDEDCAFEEEIRPDGYNVYWSKKHHLPVSLSSARQRQLYKGRGFLPLSHFLPMLSTLPAEPEDLQDPFKSDLFSLPLETDSMDPFRIAAKLGAVKSPSFYK

SEQ ID NO: 3 - protein, 218 aa, Bos taurus
MRSAPSRCAVARALVLAGLWLAAAGRPLAFSDAGPHVHYGWGESVRLRHLYTAGPQGLYSCFLRIHSDGAVDCAQVQSAHSLMEIRAVALSTVAIKGERSVLYLCMDADGKMQGLTQYSAEDCAFEEEIRPDGYNVYWSRKHHLPVSLSSSRQRQLFKSRGFLPLSHFLPMLSTIPAEPEDLQEPLKPDFFLPLKTDSMDPFGLATKLGSVKSPSFYN

SEQ ID NO: 4 - protein, 94 aa, Equus Caballus
AAGRPLALSDAGPHVHYGWGEPIRLRHLYTAGPHGLSSCFLRIRADGAVDCARGQSAHSLVEIRAVALRTVAIKGVHSVRYLCMGADGRMQGLV

SEQ ID NO: 5 - protein, 137 aa, Ovis aries
LMEIRAVALSTVAIKGERSVLFLCMDADGKMQGLTQYSAEDCAFEEEIRPDGYNVYWSRKHHLPVSLSSSRQRQLFKSRGFLPLSHFLPMLSTIPAEPEDLQEPLKPDFFLPLKTDSMDPFGLATKLGSVKSPSFYT

SEQ ID NO: 6 - protein, 193 aa, Canis familiairis
PLAFSDAGPHVHYGWGEPIRLRHLYTAGPHGLSSCFLRIRADGGVDCARGQSAHSLMEMRAVALRTVAIKGVHSGRYLCMGADGRMQGLPQYSAGDCTFEEEIRPDGYNVYWSKKHHLPISLSSAKQRQLYKGRGFLPLSHFLPILPGSPTEPRDLEDHVESDGFSASLETDSMDPFGIATKIGLVKSPSFQK

SEQ ID NO: 7 - protein, 219 aa, Felis catus
MRSAPSQCAVTRALVLAGLWLAAAGRPLAFSDAGPHVHYGWGEPIRLRHLYTAGPHGLSSCFLRIRADGGVDCARSQSAHSLVEIRAVALRTVAIKGVHSVRYLCMGADGRMQGLLQYSAGDCAFQEEIRPDGYNVYRSEKHRLPVSLSSAIQRQLYKGRGFLPLSHFLPMLPGSPAEPRDLQDHVESERFSSPLETDSMDPFGIATKMGLVKSPSFQK

SEQ ID NO: 8 - protein, 218 aa, Oryctolagus cuniculus
MRRAPSGGAAARALVLAGLWLAAAARPLALSDAGPHLHYGWGEPVRLRHLYATSAHGVSHCFLRIRADGAVDCERSQSAHSLLEIRAVALRTVAFKGVHSSRYLCMGADGRMRGQLQYSEEDCAFQEEISSGYNVYRSTTHHLPVSLSSAKQRHLYKTRGFLPLSHFLPVLPLASEETAALGDHPEADLFSPPLETDSMDPFGMATKLGPVKSPSFQK

SEQ ID NO: 9 - protein, 218 aa, Rattus norvegicus
MARKWSGRIVARALVLATLWLAVSGRPLVQQSQSVSDEGPLFLYGWGKITRLQYLYSAGPYVSNCFLRIRSDGSVDCEEDQNERNLLEFRAVALKTIAIKDVSSVRYLCMSADGKIYGLIRYSEEDCTFREEMDCLGYNQYRSMKHHLHIIFIKAKPREQLQGQKPSNFIPIFHRSFFESTDQLRSKMFSLPLESDSMDPFRMVEDVDHLVKSPSFQK

SEQ ID NO: 10 - protein, 218 aa, mus musculus
MARKWNGRAVARALVLATLWLAVSGRPLAQQSQSVSDEDPLFLYGWGKITRLQYLYSAGPYVSNCFLRIRSDGSVDCEEDQNERNLLEFRAVALKTIAIKDVSSVRYLCMSADGKIYGLIRYSEEDCTFREEMDCLGYNQYRSMKHHLHIIFIQAKPREQLQDQKPSNFIPVFHRSFFETGDQLRSKMFSLPLESDSMDPFRMVEDVDHLVKSPSFQK